(19) United States
(12) Patent Application Publication
(10) Pub. No.: US 2026/0216066 A1
(43) Pub. Date: Jul. 30, 2026

(54) FEEDING COMPOSITION

(71) Applicants: BARTS HEALTH NHS TRUST, London (GB); QUEEN MARY, UNIVERSITY OF LONDON, London (GB); GUY'S AND ST THOMAS' NHS FOUNDATION TRUST, London (GB)

(72) Inventors: Zudin PUTHUCHEARY, London (GB); Angela MCNELLY, London (GB); Danielle BEAR, London (GB); Anne LANGAN, London (GB)

(21) Appl. No.: 19/151,026

(22) PCT Filed: Dec. 15, 2023
(86) PCT No.: PCT/EP2023/086085

§ 371 Date: Jul. 25, 2025

(30) Foreign Application Priority Data
Jan. 27, 2023 (GB) 2301171.1

(51) Int. Cl. A61K 9/00; A61K 31/22; A61K 31/70; A61K 33/00; A61K 38/01

(52) U.S. Cl. CPC A61K 9/0095; A61K 9/0053; A61K 31/22; A61K 31/70; A61K 33/00; A61K 38/01

(57) ABSTRACT

A feeding composition comprising 60% to 90% by kCal contribution fat, 5% to 35% by kCal contribution protein and up to 10% by kCal contribution carbohydrate is provided for use in treating and/or preventing muscle atrophy in patients who are critically ill. The fat comprises medium chain triglycerides and long chain triglycerides. The proportion of fat comprising medium chain triglycerides is 25% to 90% by kCal contribution. The long chain triglycerides are derived from a vegetal source. The feeding composition is suitable for administration via a nasogastric tube.

The present invention relates to a feeding composition for use in treating and/or preventing muscle atrophy in patients who are critically ill and a method of preparing said feeding composition.

### BACKGROUND OF THE INVENTION

It is estimated that 195,000 patients are admitted to Intensive Care Units (ICUs) in the United Kingdom every year. Common reasons for patients requiring intensive care include serious accidents (e.g. road accidents, serious head injuries, serious falls or severe burns), serious short term conditions such as heart attack or stroke, serious infections such as sepsis, or recovery from major surgery. In the absence of care or medical intervention, they are unlikely to survive their condition. Of these patients, more than 50% are at risk of muscle atrophy i.e. muscle wasting due to derangement of metabolic processes, prolonged bed rest, immobilisation and in many cases, sedation. Currently, no satisfactory techniques exist to prevent muscle atrophy in critically ill patients.

Critically ill patients, particularly those who are unconscious, often receive enteral nutrition i.e. tube feeding as they are unable to effectively feed themselves. Typically, such feeding is nasogastric, allowing food to be carried through the nose and directly into the stomach, but can also be via gastrostomy. However, even with such feeding, critically ill patients continue to lose muscle rapidly. One reason for this may be that such patients cannot use protein or calories in the feed to maintain muscle mass.

Dietary compositions are often provided in healthcare settings to deliver specific distributions of macronutrients, vitamins and minerals. A known dietary composition is the ketogenic diet, which has been used in medicine since the 1920's and primarily consists of high amounts of fats, moderate amounts of proteins and very low amounts of carbohydrate. It has been used for treatment of the neurological disease epilepsy, particularly for paediatric patients. For example, in EP2813149 a composition comprising about 4% to about 12% by weight protein, less than 5% by weight carbohydrate and about 25% to about 38% by weight fat is disclosed for treating refractory epilepsy in children.

The ketogenic diet has also been used for treating patients with neurological injury. For example, in U.S. Pat. No. 5,308,832 a composition comprising about 70% to 85% by kCal contribution fat, 15% to 30% by kCal contribution protein and less than 5% by kCal contribution carbohydrate is disclosed. In preferred embodiments, the fat comprises medium chain triglycerides up to about 20% by kCal contribution.

In WO2015/034812, a composition comprising 50% to 80% by kCal contribution fat, 10% to 35% by kCal contribution protein and 0% to 10% by kCal contribution carbohydrate is disclosed. The fat comprises medium chain triglycerides and fish oil, which is rich in eicosapentaenoic acid (EPA) and docosahexaenoic acid (DHA) i.e. omega-3 fatty acids.

The ketogenic diet has also been recommended in the treatment of obesity and diabetes, kidney disease and cancer, but with very little evidence.

During acute illnesses, the ability to utilise carbohydrates and lipids to generate energy are greatly reduced, contributing to the loss of energy and a catabolic state. Ketogenesis provides an alternative source of energy in the form of ketone bodies, replacing glucose and lipids as a primary source of energy. Ketone bodies synthesised in the body can be easily utilised for energy production by the heart, muscle tissue, kidneys and brain. Critically ill patients still also require a source of protein.

### ASPECTS OF THE INVENTION

According to a first aspect of the invention, there is provided a feeding composition comprising 60% to 90% by kCal contribution fat, 5% to 35% by kCal contribution protein and up to 10% by kCal contribution carbohydrate for use in treating and/or preventing muscle atrophy in patients who are critically ill;
wherein the fat comprises medium chain triglycerides and long chain triglycerides;
wherein the proportion of fat comprising medium chain triglycerides is 25% to 90% by kCal contribution; and
wherein the long chain triglycerides are derived from a vegetal source.

The term “muscle atrophy” as used herein describes, but is not limited to, changes in the structure and function of the muscles. Typically, muscle atrophy is characterised by generalised muscle weakness and a loss of muscle mass.

The term “critically ill” as used herein describes, but is not limited to, patients who are either suffering from life-threatening conditions or are at risk of developing them, and who require medical intervention in the form of treatment and monitoring. Typically, critically ill patients reside in ICUs (Intensive Care Units, also known as ITUs which is short for Intensive Therapy Units), which are specialist hospital wards. These patients are often unable to mobilise either due to their condition and/or because of the equipment required for treatment, which increases the risk of muscle atrophy. Critically ill patients may also suffer from derangement of metabolic processes, where the normal functioning of metabolic processes are disrupted. Critically ill patients therefore include patients receiving intensive care, but also patients in cardiac critical care, patients in High Dependency Units (HDUs), patients in respiratory support units and neurocritically ill patients who are receiving neurocritical care. In the present invention, the feeding composition is preferably intended for patients who are presumed to survive their critically ill condition. Preferably, the patients receiving the feeding composition are adults.

The amount of fat, protein and carbohydrate present in the feeding composition is determined by kilocalorie (kCal) contribution. Fat, protein and carbohydrate generally fall under the term “macronutrients”. The proportions and amounts of macronutrients are selected to match the nutritional requirements of individual patients, whilst preferably maintaining a ketogenic diet so that patients are in a state of ketosis. Typically, the overall calorific intake for critically ill patients is based on patient weight and needs to meet a target of about 25 kCal per kilogram per day.

In the field of nutrition, energy contribution is typically given in kCal. The calorie is a unit of energy, and is equal to 4.184 J. One kCal is equal to 4.184 kJ.

The composition comprises fat. Examples of types of fat that may be used in the composition alone or in combination may include saturated fatty acids, monounsaturated fatty acids and polyunsaturated fatty acids. The types of fat are medium chain triglycerides (MCTs) and/or long chain triglycerides (LCTs). The MCTs used in the feeding composition can be derived from animal or vegetal sources.

LCTs are triglycerides having aliphatic chains of 16 to 20 carbon atoms. Examples of such aliphatic chains include oleic acid and linoleic acid. Vegetable oils typically contain LCTs with aliphatic chains comprising oleic acid and linoleic acid.

The LCTs in the feeding composition are derived from a vegetal source e.g. a vegetable oil such as rapeseed oil. Rapeseed oil typically has an oleic acid content of about 65% and a linoleic acid content of about 20% (not indicated to be determined by kCal contribution). Other known vegetable oils with similar oleic acid and linoleic acid content include avocado oil, olive oil, peanut oil and sunflower oil.

The LCTs are not derived from fish oils. Fish oils are typically rich in eicosapentaenoic acid (EPA) and docosahexaenoic acid (DHA). Both EPA and DHA are also known as omega-3 fatty acids. The feeding composition preferably does not contain fish oil, EPA or DHA.

The amount of total fat in the feeding composition is at least about 60% by kCal contribution, preferably at least about 65% by kCal contribution, and most preferably at least about 70% by kCal contribution of the feeding composition.

The total amount of fat in the feeding composition is up to about 90% by kCal contribution. However, in some embodiments the amount of total fat in the feeding composition is no more than about 85% by kCal contribution, e.g. no more than about 80% by kCal contribution of the feeding composition.

The total amount of fat in the feeding composition is in a range from about 60% to about 90% by kCal contribution, preferably from about 65% to about 85% by kCal contribution, most preferably from about 70% to about 80% by kCal contribution. In one embodiment, the feeding composition comprises about 75% total fat by kCal contribution. In another embodiment, the feeding composition comprises about 70% total fat by kCal contribution.

The amounts of total fat, MCTs, protein and carbohydrate in the feeding composition may alternatively be determined as percentages by weight.

As percentages by weight (proportion of fat (total), protein and carbohydrate), the amount of total fat in the feeding composition is at least about 40% by weight, preferably at least about 45% by weight of the feeding composition. The total amount of fat in the feeding composition is up to about 65% by weight. However, in some embodiments the amount of total fat in the feeding composition is no more than about 60% by weight of the feeding composition.

Preferably, the total amount of fat in the feeding composition (proportion of fat (total), protein and carbohydrate) is in a range from about 40% to about 65% by weight, preferably from about 45% to about 60% by weight. In one embodiment, the feeding composition comprises about 50% by weight total fat. In another embodiment, the feeding composition comprises about 56% by weight total fat.

At least one type of fat present in the feeding composition is MCTs. These are triglycerides having shorter aliphatic chains of 6 to 12 carbon atoms. Examples of such aliphatic chains include caproic acid, caprylic acid, capric acid and lauric acid. Common sources of MCTs include palm kernel oil and coconut oil. Advantageously, when a high proportion of the total fat in the feeding composition is MCTs, ketogenesis can be induced. The amount of MCTs in the feeding composition, that is to say the proportion of the total fat that is MCTs, is also determined by kCal contribution.

The proportion of total fat that is MCTs in the feeding composition is at least about 25% by kCal contribution, preferably at least about 30% by kCal contribution, and most preferably at least about 35% by kCal contribution.

In some embodiments, the proportion of total fat that is MCTs is up to about 90% by kCal contribution. However, in other embodiments the proportion of total fat that is MCTs in the feeding composition is no more than about 85% by kCal contribution, e.g. no more than about 80% by kCal contribution.

The proportion of total fat that is MCTs in the feeding composition is typically in a range from about 25% to about 90% by kCal contribution, preferably from about 30% to about 85% by kCal contribution, most preferably from about 35% to about 80% by kCal contribution.

High fat sedatives are frequently administered to critically ill patients in ICUs. An example of a high fat sedative used in ICUs is propofol, which contributes significantly to the total daily amount of fat a patient receives because it is usually formulated as an emulsion containing oils, such as soya-bean oil.

High fat sedatives are typically administered via peripheral intravenous (IV) therapy. The amount of high fat sedative administered varies between patients and is not just a function of patient weight. Typically, critically ill patients will receive an initial dose of high fat sedative, with the dose subsequently decreasing over time. Therefore, the amount of MCTs in the feeding composition can be altered to meet the desired nutritional requirements of an individual patient, whilst also maintaining a ketogenic diet.

In these embodiments, the proportion of total fat that is MCTs in the feeding composition is usually at least about 55% by kCal contribution, preferably at least about 60% by kCal contribution, and most preferably at least about 65% by kCal contribution. The proportion of total fat that is MCTs in these embodiments is up to about 90% by kCal contribution. However, in other embodiments the proportion of total fat that is MCTs in the feeding composition is no more than about 85% by kCal contribution, e.g. no more than about 80% by kCal contribution. In these embodiments, the proportion of total fat that is MCTs is in a range from about 55% to about 90% by kCal contribution, preferably from about 60% to about 85% by kCal contribution, most preferably from about 65% to about 80% by kCal contribution.

In a particularly preferred embodiment where critically ill patients are receiving high fat sedative, the proportion of total fat in the feeding composition that is MCTs is 70% by kCal contribution.

In a particularly preferred embodiment where critically ill patients are not receiving high fat sedative, the proportion of total fat in the feeding composition that is MCTs is 76% by kCal contribution.

As a proportion of kCals from total fat, protein and carbohydrate, the amount of MCTs in the feeding composition is at least about 40% by kCal contribution, preferably at least about 45% by kCal contribution. However, in some embodiments the amount of MCTs as a proportion of kCals from total fat, protein and carbohydrate is no more than 65% by kCal contribution, e.g. no more than about 60% by kCal contribution.

Preferably, the amount of MCTs as a proportion of kCals from total fat, protein and carbohydrate is in a range from about 40% to about 65% by kCal contribution, preferably from about 45% to about 60% by kCal contribution.

In a particularly preferred embodiment where critically ill patients are receiving high fat sedative, the proportion of kCals from total fat, protein and carbohydrate in feeding composition that is MCTs is 50% by kCal contribution.

In a particularly preferred embodiment where critically ill patients are not receiving high fat sedative, the proportion of kCals from total fat, protein and carbohydrate in feeding composition that is MCTs is 57% by kCal contribution.

As percentages by weight (proportion of fat (total), protein and carbohydrate), the amount of MCTs in the feeding composition is at least about 30% by weight, preferably at least about 35% by weight of the feeding composition. Up to about 55% by weight of the feeding composition may be MCTs. However, in some embodiments the amount of MCTs in the feeding composition is no more than about 50% by weight of the feeding composition.

Preferably, the amount of MCTs in the feeding composition (proportion of fat (total), protein and carbohydrate) is in a range from about 30% to about 55% by weight, preferably from about 35% to about 50% by weight. In one preferred embodiment, the feeding composition comprises about 38% by weight MCTs. In another preferred embodiment, the feeding composition comprises about 45% by weight MCTs.

Another option for altering the amount of MCTs to meet the desired nutritional requirements of an individual patient who is receiving, or not receiving, high fat sedative whilst also maintaining a ketogenic diet is to adjust the volume of the feeding composition that is administered to the critically ill patient.

The amount of feeding composition administered to each critically ill patient is individualised to reflect not only the patient's body mass index, but also clinical status.

In embodiments where critically ill patients are receiving high fat sedative, the volume of the feeding composition administered to the critically ill patient is usually at least about 300 mL, preferably at least about 375 mL, and most preferably at least about 450 mL.

In embodiments where critically ill patients are receiving high fat sedative, the volume of the feeding composition administered to the critically ill patient may be up to about 800 mL. However, in other embodiments the volume of the feeding composition administered to the critically ill patient is no more than about 725 mL, e.g. no more than about 650 mL.

In embodiments where critically ill patients are receiving high fat sedative, the volume of the feeding composition administered to the critically ill patient is typically in a range from about 300 mL to about 800 mL, preferably from about 375 mL to about 725 mL, most preferably from about 450 mL to about 650 mL. In these embodiments, the feeding composition supplements the macronutrients the critically ill patients receive from the high fat sedative.

In embodiments where critically ill patients are not receiving high fat sedative, the volume of the feeding composition administered to the critically ill patient is usually at least about 600 mL, preferably at least about 675 mL, and most preferably at least about 750 mL.

In embodiments where critically ill patients are not receiving high fat sedative, the volume of the feeding composition administered to the critically ill patient may be up to about 1400 mL. However, in other embodiments the volume of the feeding composition administered to the critically ill patient is no more than about 1325 mL, e.g. no more than about 1250 mL.

In embodiments where critically ill patients are not receiving high fat sedative, the volume of the feeding composition administered to the critically ill patient is typically in a range from about 600 mL to about 1400 mL, preferably from about 675 mL to about 1325 mL, most preferably from about 750 mL to about 1250 mL. In these embodiments, the feeding composition supplies at least the majority e.g. all of the macronutrients supplied to the critically ill patients. Therefore, the amount of the feeding composition administered to the critically ill patients not receiving high fat sedative is typically more when compared to the amount administered to critically ill patients receiving high fat sedative.

In an alternative embodiment where critically ill patients are receiving high fat sedative, the proportion of total fat in the feeding composition that is MCTs is in a range from about 40% to about 50% by kCal contribution. In a particular alternative embodiment where critically ill patients are receiving high fat sedative, the proportion of total fat in the feeding composition that is MCTs is 47% by kCal contribution.

In other alternative embodiments, the proportion of total fat that is MCTs ranges from about 60% to about 90% by kCal contribution, e.g. in a range from about 65% to about 85% by kCal contribution. These embodiments are alternative embodiments for patients not receiving high fat sedative. In an alternative embodiment where critically ill patients are not receiving high fat sedative, the proportion of total fat in the feeding composition that is MCTs is in a range from about 70% to about 80% by kCal contribution. In a particular alternative embodiment where critically ill patients are not receiving high fat sedative, the proportion of total fat in feeding composition that is MCTs is 75% by kCal contribution.

In preferred embodiments, the fat and MCT source used in the feeding composition includes K.Quik™ (formerly BETAQUIK™) and/or FRESUBIN™ 5 kcal Shot. The amounts of energy and macronutrients present in K.Quik™ and FRESUBIN™ are provided in Table 1.

TABLE 1

| | K.Quik ™ | FRESUBIN ™ 5 kcal Shot |
|---|---|---|
| | (per 100 mL) | (per 100 mL) |
| Energy (kJ) | 777 | 2100 |
| Energy (kCal) | 189 | 500 |
| Total fat (g) | 21 | 53.8 |
| MCT fat (g) | 20 | 13.9 |
| Protein (g) | 0 | 0 |
| Carbohydrate (g) | 0 | 4 |

The MCTs in K.Quik™ are from coconut oil and palm kernel oil.

The LCTs in FRESUBIN™ 5 kcal Shot are from rapeseed oil.

The composition also comprises protein. Proteins are macronutrients made up of amino acids and can be derived from both animal and vegetal based sources. Typically, the amount of protein required by critically ill patients who are nasogastrically fed is 0.88-1.25 g per kg per day, based on an individual patient's weight. The amount of protein required in the feeding composition is also determined by kCal contribution.

The amount of protein in the feeding composition is at least about 5% by kCal contribution, preferably at least about 10% by kCal contribution of the feeding composition.

Up to about 35% by kCal contribution of the feeding composition is protein. However, in some embodiments the amount of protein in the feeding composition is no more than about 30% by kCal contribution of the feeding composition.

The amount of protein in the feeding composition is in a range from about 5% to about 35% by kCal contribution, preferably from about 10% to about 30% by kCal contribution. In one embodiment, the feeding composition comprises about 20% by kCal contribution protein. In another embodiment, the feeding composition comprises about 26% by kCal contribution protein.

As percentages by weight (proportion of fat (total), protein and carbohydrate), the amount of protein in the feeding composition is usually at least about 25% by weight, preferably at least about 30% by weight of the feeding composition. Up to about 50% by weight of the feeding composition may be protein. However, in some embodiments the amount of protein in the feeding composition is no more than about 45% by weight of the feeding composition.

The amount of protein in the feeding composition (proportion of fat (total), protein and carbohydrate) is typically in a range from about 25% to about 50% by weight, preferably from about 30% to about 45% by weight. In one preferred embodiment, the feeding composition comprises about 33% by weight protein. In another preferred embodiment, the feeding composition comprises about 41% by weight protein.

In preferred embodiments, the source of protein used in the feeding composition is RENAPRO™ Shot, which comprises bovine collagen peptides. The amounts of energy and macronutrients present in RENAPRO™ are provided in Table 2.

TABLE 2

| | RENAPRO ™ Shot |
|---|---|
| | (per 100 mL) |
| Energy (kJ) | 711 |
| Energy (kCal) | 167 |
| Total fat (g) | <0.5 |
| Protein (g) | 33 |
| Carbohydrate (g) | 4.9 |

The feeding composition also comprises carbohydrate. Preferably, the feeding composition comprises carbohydrate in a relatively low amount compared to the fat and protein to provide a ketogenic diet i.e. the amount of carbohydrate is preferably lower than the amount of fat and protein in the feeding composition.

Preferably, the carbohydrate sources that may be used in the composition alone or in combination includes polysaccharides, oligosaccharides, disaccharides, and monosaccharides. A particularly preferred type of carbohydrate is monosaccharide, for example glucose. In one embodiment, the carbohydrate source in the feeding composition includes Super Soluble Maxijul™, which comprises dried glucose syrup.

The amounts of energy and macronutrients present in Super Soluble Maxijul™ are provided in Table 3.

TABLE 3

| | Super Soluble Maxijul ™ |
|---|---|
| | (per 100 g) |
| Energy (kJ) | 380 |
| Energy (kCal) | 1615 |
| Total fat (g) | 0 |
| Protein (g) | 0 |
| Carbohydrate (g) | 95 |

Carbohydrate is present in an amount of up to about 10% by kCal contribution of the feeding composition, more preferably in an amount up to about 8% by kCal contribution of the feeding composition and most preferably in an amount up to about 6% by kCal contribution of the feeding composition. In one preferred embodiment, the feeding composition comprises about 5% by kCal contribution carbohydrate. In another preferred embodiment, the feeding composition comprises about 3% by kCal contribution carbohydrate.

As percentages by weight (proportion of fat (total), protein and carbohydrate), the carbohydrate is preferably present in an amount of up to about 15% by weight of the feeding composition, more preferably in an amount up to about 13% by weight of the feeding composition. In one preferred embodiment, the feeding composition comprises about 9% by weight carbohydrate. In another preferred embodiment, the feeding composition comprises about 11% by weight carbohydrate.

In one particularly preferred embodiment according to the first aspect, the feeding composition comprises about 70% to about 75% by kCal contribution fat (of which about 70% to about 76% by kCal contribution is MCTs), about 20% to about 26% by kCal contribution protein and about 3% to about 5% by kCal contribution carbohydrate.

As percentages by weight (proportion of fat (total), protein and carbohydrate), the feeding composition comprises about 50% to about 56% by weight fat, about 38% to about 45% by weight MCTs, about 33% to about 41% by weight protein and about 9% to about 11% by weight carbohydrate.

The composition may also further comprise micronutrients including vitamins, minerals and/or trace elements. Minerals and trace elements may include, but are not limited to, sodium, potassium, chloride, calcium, phosphorus, iron, magnesium and zinc. Vitamins may include, but are not limited to, Vitamin A, Vitamin B (including B1 (thiamin), B2 (riboflavin), B3 (niacin), B5 (pantothenic acid), B6 (pyridoxine), B7 (biotin), B9 (folate) and B12 (cobalamin)), Vitamin C, Vitamin D, Vitamin E and Vitamin K.

Preferably, the feeding composition is provided to the patient by enteral feeding, which means that the feeding composition is supplied directly into the gastrointestinal tract. Examples of enteric feeding include providing the feeding composition via a nasogastric tube (using a thin soft tube passed through the nose, down the back of the throat, through the oesophagus and into the stomach) or a gastrostomy tube (a feeding tube which is inserted endoscopically or surgically through the abdominal wall and directly into the stomach). The apparatus used for enteral feeding is known as a “giving set”, which typically comprises a feeding tube with suitable attachments for connection to a feed bag. Enteral feeding is advantageous because critically ill patients are usually unable to be fed through the mouth due to their condition or because of equipment such as ventilators. Most preferably, the feeding composition is provided via a nasogastric tube.

Alternatively, the feeding composition may be provided to patients via oral administration e.g. as a drink. Embodiments where the feeding composition is fed via oral administration are particularly advantageous for patients who are not sedated and have sufficient ability to swallow the feeding composition. Providing the feeding composition in this form, where suitable, allows the feeding composition to be provided in a less invasive manner.

Preferably, the feeding composition is in a flowable liquid state. By providing the feeding composition in a flowable liquid state, the feeding composition is suitable for passing through a feeding tube of a giving set e.g. a nasogastric tube or gastrostomy tube. A flowable liquid state also allows the feeding composition to be administered as a drink. Water may be present in the feeding composition to form the flowable liquid feeding composition.

The amount of water may be up to about 85% by volume of the feeding composition, more preferably in an amount up to about 80% by volume of the feeding composition and most preferably in an amount up to about 75% by volume of the feeding composition.

The feeding composition is preferably fed continuously. The rate of continuous feeding is determined by the individual patient's weight, with a target of feeding rate of 25 kCal per kilogram per day. Typically, the feed rate of the feeding composition is up to about 70 mL per hour to about 80 mL per hour. When critically ill patients in ICUs are taken away for periods of time to have medical scans, treatments and/or surgery etc., it is often not possible to provide the feeding composition to the patient. In such scenarios, the patient may receive a top up of the feeding composition when they return to the ICU, which is achieved by administering the feeding composition at an increased feed rate to reach the daily kCal target for that patient.

Alternatively, the feeding composition is fed intermittently as a bolus. If the bolus is fed by oral administration, then the bolus is preferably fed in the form of a drink.

In embodiments where the feeding composition is fed as a bolus or as a drink, the feeding composition is preferably fed to the patient 3 to 6 times a day. In these embodiments, the amount of feeding composition provided is also determined by the individual patient's weight, with a target of feeding rate of 25 kCal per kilogram per day.

Less preferred examples of feeding compositions according are provided in Tables 4 and 5.

TABLE 4

| Macronutrient Component | % kCal Contribution | % by weight |
|---|---|---|
| Fat (total) | 75 | 15 |
| MCTs (proportion of total fat) | 47 | 12 |
| Protein | 20 | 13 |
| Carbohydrate | 5 | 3 |

TABLE 5

| Macronutrient Component | % kCal Contribution | % by weight |
|---|---|---|
| Fat (total) | 75 | 17 |
| MCTs (proportion of total fat) | 75.5 | 14 |
| Protein | 20 | 10 |
| Carbohydrate | 5 | 3 |

According to a second aspect of the invention, there is provided a method of preparing a feeding composition, comprising:
mixing a feeding composition according to the first aspect of the invention in a vessel;
and optionally
decanting the mixture into a feed bag, wherein the feed bag is optionally provided with a giving set.

The feeding composition may be used immediately after preparation or may be stored at a temperature of 0° C. to 5° C., until required for feeding.

As percentages by weight (proportion of fat (total), protein and carbohydrate), there is provided a method of preparing a feeding composition according to the second aspect, comprising mixing 40% to 65% by weight fat, 25% to 50% by weight protein and up to 15% by weight carbohydrate in a vessel and optionally decanting the mixture into a feed bag, wherein the feed bag is optionally provided with a giving set. The feeding composition may be used immediately after preparation or may be stored at a temperature of 0° C. to 5° C., until required for feeding.

Without wishing to be bound by theory, the inventors believe that the ketones induced by the MCTs in the ketogenic feeding composition provide another substrate for cells to produce energy from, instead of protein. Carbohydrate metabolism switches to anaerobic in the critically ill via the Pasteur effect, which produces much less adenosine triphosphate (ATP). Fatty acid ß-oxidation, the process by which fatty acids are broken down to produce energy, is blocked.

Features described in connection with one aspect of the invention can be used in connection with any aspect of the invention.

### DETAILED DESCRIPTION OF THE INVENTION

The invention will now be described by way of the following non-limiting examples.

### Example 1—Patients Receiving Propofol

The proportions by % kCal contribution and % by weight of total fat, MCTs, protein and carbohydrate in an exemplary feeding composition according to the present invention for patients receiving propofol are provided in Tables 6 and 7.

TABLE 6

| Macronutrient Component | % kCal Contribution |
|---|---|
| Fat (total) | 70.4 |
| MCTs (as proportion of total fat) | 70 |
| MCTs (as proportion of total kCals from | 49.5 |
| fat (total), protein and carbohydrate) | |
| Protein | 26.3 |
| Carbohydrate | 3.3 |

In Table 6, the combined % kCal contribution of fat (total), protein and carbohydrate is equal to 100% kCal contribution. The % kCal contribution of MCTs as a proportion of total fat is 70%, i.e. 70% of the kCal contribution of fat is from MCTs. The % kCal contribution of MCTs as a proportion of total kCals from fat (total), protein and carbohydrate is 49.5%, i.e. 49.5% of the total kCals in the feed is from MCTs. If patients receive an average daily volume of propofol of 367 mL, then 46% of the kCal contribution of fat is from MCTs i.e. the amount of MCTs the patient receives from the feeding composition and propofol together is 46% kCal contribution.

TABLE 7

| Macronutrient Component | % by Weight |
|---|---|
| Fat (total) | 49.7 |
| MCTs (proportion of fat (total), protein and carbohydrate) | 38.1 |
| Protein | 41.4 |
| Carbohydrate | 8.9 |

In Table 7, the combined % by weight of fat (total), protein and carbohydrate is equal to 100% by weight. The combined % by weight of fat (total), protein and carbohydrate therefore excludes other components in the feeding composition e.g. water. The % by weight of MCTs is not % by weight proportion of total fat, but instead the % by weight proportion of the combined % by weight of fat (total), protein and carbohydrate. The water content is around 70% by volume.

The ingredients used to obtain the feeding composition in Tables 6 and 7 are provided in Table 8. In an ICU pantry, the ingredients were measured out in a measuring jug and mixed together within the jug using a sterile spoon. The resulting composition was then decanted into a feed bag provided with a giving set and stored in a refrigerator until required.

TABLE 8

| Ingredient | Amount | |
|---|---|---|
| K.Quik ™ | 300.5 | mL |
| RENAPRO ™ | 217.5 | mL |
| Super Soluble Maxijul | 3.15 | g |
| FRESUBIN ™ 5 kcal Shot | 43 | mL |

The total volume of the feeding composition is 561 mL.

### Example 2—Patients not Receiving Propofol

The proportions by % kCal contribution and % by weight of total fat, MCTs, protein and carbohydrate in an exemplary feeding composition according to the present invention for patients not receiving propofol are provided in Tables 9 and 10.

TABLE 9

| Macronutrient Component | % kCal Contribution |
|---|---|
| Fat (total) | 75.4 |
| MCTs (as proportion of total fat) | 75.5 |
| MCTs (as proportion of total kCals from | 57.1 |
| fat (total), protein and carbohydrate) | |
| Protein | 19.6 |
| Carbohydrate | 5.0 |

In Table 9, the combined % kCal contribution of fat (total), protein and carbohydrate is equal to 100% kCal contribution. The % kCal contribution of MCTs as a proportion of total fat is 75.5%, i.e. 75.5% of the kCal contribution of fat is from MCTs. The % kCal contribution of MCTs as a proportion of total kCals from fat (total), protein and carbohydrate is 57.1%, i.e. 57.1% of the total kCals in the feed is from MCTs.

TABLE 10

| Macronutrient Component | % by Weight |
|---|---|
| Fat (total) | 56.1 |
| MCTs (proportion of fat (total), protein and carbohydrate) | 45.4 |
| Protein | 32.7 |
| Carbohydrate | 11.2 |

In Table 10, the combined % by weight of fat (total), protein and carbohydrate is equal to 100% by weight. The combined % by weight of fat (total), protein and carbohydrate therefore excludes other components in the feeding composition e.g. water. The % by weight of MCTs is not % by weight proportion of total fat, but instead the % by weight proportion of the combined % by weight of fat (total), protein and carbohydrate. The water content is around 70% by volume.

The ingredients used to obtain the feeding composition in Tables 9 and 10 are provided in Table 11. The composition was prepared as in Example 1.

TABLE 11

| Ingredient | Amount | |
|---|---|---|
| K.Quik ™ | 639.5 | mL |
| RENAPRO ™ | 298.5 | mL |
| Super Soluble Maxijul | 17.35 | g |
| FRESUBIN ™ 5 kcal Shot | 65 | mL |

The total volume of the feeding composition is 1003 mL.

The feeding compositions of Example 1 and Example 2 were fed to a group of 29 patients in ICUs to meet an individualised target feeding rate of 25 kCal per kilogram per day. Patients receiving propofol were fed the composition of Example 1, whilst patients not receiving propofol were fed the composition of Example 2. Any changes to a patient's routine, such as medical scans, therapy or surgery etc. were recorded and the feed rate increased as a “top up” afterwards to achieve the target daily calorie intake. The amount of feeding composition given per day to each patient was monitored and mean values were taken to provide a feeding composition for future use. The feeding composition was found to put the patients in a state of ketosis.

Table 12 shows the mean (from the upper and lower levels) % kCal contribution of MCTs given to the patients over a 10 day period. The patients received the feeding composition whilst also receiving propofol. The dose of propofol decreased over the 10 day period. The mean (from the upper and lower levels) % kCal contribution of MCTs values are the amount of MCTs the patients received from the feeding composition and propofol together by % kCal contribution of fat. The % kCal contribution of MCTs was increased from a mean of 44.41% kCal contribution to a mean of 73.96% kCal contribution whilst monitoring the patients to ensure that the % kCal contribution of MCTs were safe, tolerated and feasible to give to a critically ill population. It was determined following patient monitoring that the % kCal contribution of MCTs administered to the patient group was safe.

TABLE 12

| | Day 1 | Day 2 | Day 3 | Day 4 | Day 5 | Day 6 | Day 7 | Day 8 | Day 9 | Day 10 |
|---|---|---|---|---|---|---|---|---|---|---|
| Mean (%) | 44.41 | 53.63 | 62.05 | 69.14 | 77.13 | 76.25 | 76.12 | 74.5 | 73.9 | 73.96 |
| Upper | 52.8 | 61.85 | 69.89 | 78.43 | 86.13 | 86.36 | 88.12 | 87.26 | 84.27 | 83.77 |
| level (%) | | | | | | | | | | |
| Lower | 36.02 | 45.41 | 54.21 | 59.85 | 68.13 | 66.14 | 64.12 | 61.74 | 63.53 | 64.15 |
| level (%) | | | | | | | | | | |

Aspects of the invention include:
#1. A feeding composition comprising 60% to 90% by kCal contribution fat, 5% to 35% by kCal contribution protein and up to 10% by kCal contribution carbohydrate for use in treating and/or preventing muscle atrophy in patients who are critically ill.
#2. A feeding composition according to #1, wherein the composition is suitable for oral administration and/or enteral feeding e.g. via gastrostomy or nasogastric administration.
#3. A feeding composition according to either #1 or #2, wherein the patients are adults.
#4. A feeding composition according to any preceding aspect, comprising 75% by kCal contribution fat.
#5. A feeding composition according to any preceding aspect, wherein the fat comprises medium chain triglycerides.
#6. A feeding composition according to #5, wherein the proportion of fat comprising medium chain triglycerides is preferably 25% to 90% by kCal contribution, more preferably 35% to 60% by kCal contribution, and most preferably 40% to 55% by kCal contribution.
#7. A feeding composition according to #5, wherein the proportion of fat comprising medium chain triglycerides is 60% to 90% by kCal contribution, preferably 65% to 85% by kCal contribution.
#8. A feeding composition according to #5, wherein the proportion of fat comprising medium chain triglycerides is 47% by kCal contribution.
#9. A feeding composition according to #5, wherein the proportion of fat comprising medium chain triglycerides is 75% by kCal contribution.
#10. A feeding composition according to any preceding aspect, comprising 20% by kCal contribution protein.
#11. A feeding composition according to any preceding aspect, comprising 5% by kCal contribution carbohydrate.
#12. A feeding composition according to any preceding aspect, further comprising micronutrients selected from vitamins, minerals and trace elements.
#13. A feeding composition comprising 75% by kCal contribution fat, 20% by kCal contribution protein and 5% by kCal contribution carbohydrate.
#14. A feeding composition according to #13, wherein the fat comprises medium chain triglycerides.
#15. A feeding composition according to #14, wherein the proportion of fat comprising medium chain triglycerides is 47% by kCal contribution.
#16. A feeding composition according to #14, wherein the proportion of fat comprising medium chain triglycerides is 75% by kCal contribution.
#17. A method of preparing a feeding composition, comprising:
mixing 60% to 90% by kCal contribution fat, 5% to 35% by kCal contribution protein and up to 10% by kCal contribution carbohydrate in a vessel; and optionally decanting the mixture into a feed bag, wherein the feed bag is optionally provided with a giving set.
#18. A method according to #17, further comprising storing the mixture at a temperature of 0° C. to 5° C.
#19. A feeding composition comprising 9% to 21% by weight fat, 6% to 17% by weight protein and up to 5% by weight carbohydrate for use in treating and/or preventing muscle atrophy in patients who are critically ill.
#20. A feeding composition according to #19, comprising 15% or 17% by weight fat.
#21. A feeding composition according to any of #19 to #20, wherein the fat comprises medium chain triglycerides.
#22. A feeding composition according to #21, comprising 8% to 18% by weight medium chain triglycerides.
#23. A feeding composition according to either #21 or #22, comprising 12% or 14% by weight medium chain triglycerides.
#24. A feeding composition according to any of #19 to #23, comprising 10% or 13% by weight protein.
#25. A feeding composition according to any of #19 to #24, comprising 3% by weight carbohydrate.

1. A feeding composition comprising 60% to 90% by kCal contribution fat, 5% to 35% by kCal contribution protein and up to 10% by kCal contribution carbohydrate for use in treating and/or preventing muscle atrophy in patients who are critically ill;
wherein the fat comprises medium chain triglycerides and long chain triglycerides;
wherein the proportion of fat comprising medium chain triglycerides is 25% to 90% by kCal contribution; and
wherein the long chain triglycerides are derived from a vegetal source.

2. A feeding composition according to claim 1, wherein the vegetal source is rapeseed oil.

3. A feeding composition according to claim 1 or 2, wherein the fat is 65% to 85% by kCal contribution, preferably 70% to 80% by kCal contribution.

4. A feeding composition according to any preceding claim, wherein the proportion of fat comprising medium chain triglycerides is 55% to 90% by kCal contribution, preferably 60% to 85% by kCal contribution.

5. A feeding composition according to any preceding claim, wherein the protein is 10% to 30% by kCal contribution.

6. A feeding composition according to any preceding claim, wherein the carbohydrate is up to 8% by kCal contribution, preferably up to 6% by kCal contribution.

7. A feeding composition according to any preceding claim, further comprising micronutrients selected from vitamins, minerals and trace elements.

8. A feeding composition according to any preceding claim, wherein the composition is suitable for oral administration and/or enteral feeding e.g. via gastrostomy or nasogastric administration.

9. A feeding composition according to any preceding claim, wherein the patients are adults.

10. A feeding composition according to any preceding claim, wherein the patients are receiving high fat sedative.

11. A feeding composition according to any of claims 1 to 9, wherein the patients are not receiving high fat sedative.

12. A feeding composition according to any preceding claim, comprising 70% by kCal contribution fat, 26% by kCal contribution protein and 3% by kCal contribution carbohydrate;
wherein the proportion of fat comprising medium chain triglycerides is 70% by kCal contribution.

13. A feeding composition according to any preceding claim, comprising 75% by kCal contribution fat, 20% by kCal contribution protein and 5% by kCal contribution carbohydrate;
wherein the proportion of fat comprising medium chain triglycerides is 76% by kCal contribution.

14. A method of preparing a feeding composition, comprising:
mixing a feeding composition according to any of claims 1 to 13 in a vessel;
and optionally
decanting the mixture into a feed bag, wherein the feed bag is optionally provided with a giving set.

15. A method according to claim 14, further comprising storing the mixture at a temperature of 0° C. to 5° C.
